(19) United States
(12) Patent Application Publication
(10) Pub. No.: US 2025/0381176 A1
(43) Pub. Date: Dec. 18, 2025

(54) SCD1 INHIBITORS FOR TREATING LIVER DISEASE

(71) Applicant: MODULATION THERAPEUTICS, INC., Morgantown, WV (US)

(72) Inventors: Lori HAZLEHURST, Morgantown, WV (US); Sridhar KAULAGARI, Morgantown, WV (US); Karen HAYES, Morgantown, WV (US)

(21) Appl. No.: 18/877,159

(22) PCT Filed: Jun. 27, 2023
(86) PCT No.: PCT/US2023/026371

§ 371 Date: Dec. 19, 2024

Related U.S. Application Data
(60) Provisional application No. 63/356,419, filed on Jun. 28, 2022.

(51) Int. Cl. A61K 31/4545; A61K 31/4439; A61P 1/16; C07B 59/00; C07D 401/12

(52) U.S. Cl. CPC A61K 31/4545; A61K 31/4439; A61P 1/16; C07B 59/002; C07D 401/12

(57) ABSTRACT

Methods for treating non-alcoholic fatty liver disease, non-alcoholic steatohepatitis, and related diseases and disorders with an SCD 1 inhibitor (e.g., Compound 1) or a pharmaceutical salt, tautomer, stereoisomer, prodrug, or isotopologue thereof. Methods associated with preparation of pharmaceutical compositions comprising such SCD 1 inhibitor compounds are also provided.

### BACKGROUND

### Technical Field

Embodiments of the present disclosure are generally directed to use of SCD1 inhibitor compounds as therapeutic or prophylactic agents, for example for treatment of liver diseases and disorders (e.g., non-alcoholic fatty liver disease and/or non-alcoholic steatohepatitis).

### Description of the Related Art

Liver damage from non-alcoholic fatty liver disease (NAFLD) is one of the most common liver diseases in children and adults affecting 25% of the global population, with this disease leading to either simple hepatic steatosis or cirrhosis of the liver, which results in increased morbidity and mortality. Non-alcoholic steatohepatitis (NASH) is a chronic and progressive disease and is considered an advanced form of NAFLD. A common clinical manifestation of obesity is NAFLD/NASH as well as other metabolic syndromes such as diabetes. While several molecular mechanisms have been identified in contributing to NAFLD, deleterious accumulation of lipids in the liver play a significant role. With the increase worldwide of metabolic syndromes such as obesity and diabetes, finding a treatment strategy to prevent or reverts NAFLD and progression to NASH is a critical gap that remains to be fully elucidated.

Liver transplant is the only current treatment method for liver cirrhosis and NASH, thus there is a critical need for pharmaceutical modifiers of the disease progression from the early fatty liver stage before progression to cirrhosis. Reduction of liver lipid triglyceride levels have been associated with improved liver function and attenuation of NAFLD.

Accordingly, there is a need to develop SCD1 inhibitors that can be used to treat NAFLD and NASH. Embodiments of the present disclosure fulfill this need and provide further related advantages.

### BRIEF SUMMARY

In brief, embodiments of the present disclosure provide compounds, including pharmaceutically acceptable salts, stereoisomers, and prodrugs thereof, which can be used for treating various liver diseases (e.g., NAFLD and/or NASH).

One embodiment provides a method for treating non-alcoholic fatty liver disease, the method comprising administering an effective amount of a compound of Structure (I):

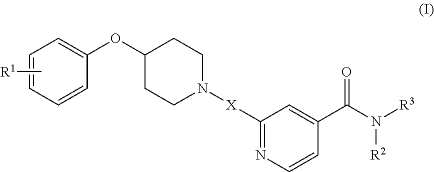

or a pharmaceutical salt, tautomer, stereoisomer, prodrug, or isotopologue thereof,
wherein:
   X is —(C═O)NR⁴—;
   R¹ is halo; and
   R², R³, and R⁴ are each independently hydrogen or an unsubstituted C₁₋₆ alkyl, to a subject in need thereof.

Another embodiment provides a method of treating non-alcoholic steatohepatitis, the method comprising administering an effective amount of a compound of Structure (I):

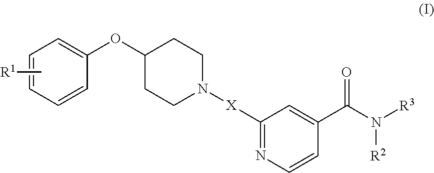

or a pharmaceutical salt, tautomer, stereoisomer, prodrug, or isotopologue thereof,
wherein:
   X is —(C═O)NR⁴—;
   R¹ is halo; and
   R², R³, and R⁴ are each independently hydrogen or an unsubstituted C₁₋₆ alkyl, to a subject in need thereof.

Still another embodiment provides a method of reducing excess fat build up in the liver of a subject, the method comprising administering an effective amount of a compound of Structure (I):

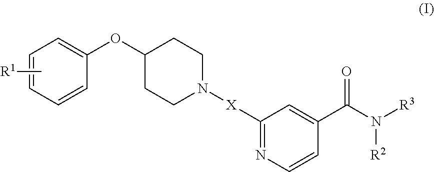

or a pharmaceutical salt, tautomer, stereoisomer, prodrug, or isotopologue thereof,
wherein:
   X is —(C═O)NR⁴—;
   R¹ is halo; and
   R², R³, and R⁴ are each independently hydrogen or an unsubstituted C₁₋₆ alkyl, to a subject in need thereof.

One embodiment provides a method for reducing the number of lipids in hepatic cells of a subject, the method comprising administering an effective amount of a compound of Structure (I):

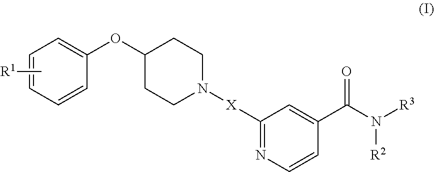

or a pharmaceutical salt, tautomer, stereoisomer, prodrug, or isotopologue thereof,
wherein:
   X is —(C═O)NR⁴—;
   R¹ is halo; and
   R², R³, and R⁴ are each independently hydrogen or an unsubstituted C₁₋₆ alkyl, to a subject in need thereof.

Another embodiment provides a method for reducing the number of lipids in a hepatic cell, the method comprising contacting the hepatic cell with an effective amount of a compound of Structure (I):

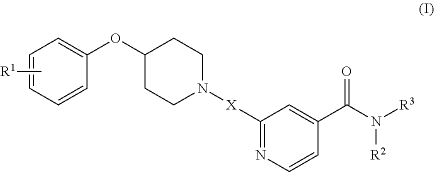

or a pharmaceutical salt, tautomer, stereoisomer, prodrug, or isotopologue thereof,
wherein:
   X is —(C═O)NR⁴—;
   R¹ is halo; and
   R², R³, and R⁴ are each independently hydrogen or an unsubstituted C₁₋₆ alkyl, to the hepatic cells.

### BRIEF DESCRIPTION OF THE SEVERAL VIEWS OF THE DRAWINGS

In the figures, identical reference numbers identify similar elements. The sizes and relative positions of elements in the figures are not necessarily drawn to scale and some of these elements are enlarged and positioned to improve figure legibility. Further, the particular shapes of the elements as drawn are not intended to convey any information regarding the actual shape of the particular elements and have been solely selected for ease of recognition in the figures.

FIG. 1 shows a schematic representation of the pathway in which the SCD1 enzyme plays an important role in liver lipid accumulation. Inhibition/downregulation of the SCD1 shifts flow of lipids from damaging liver storage to beneficial oxidative metabolism pathways (vertical arrows to the right); SCD1 inhibition is an emerging therapeutic pathway for anti-steatosis treatment.

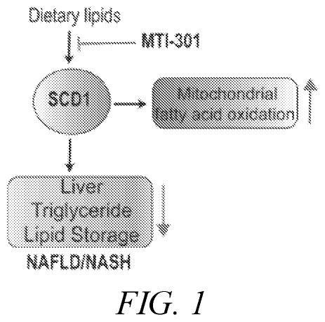

FIG. 2A shows the chemical structure of Compound 1.

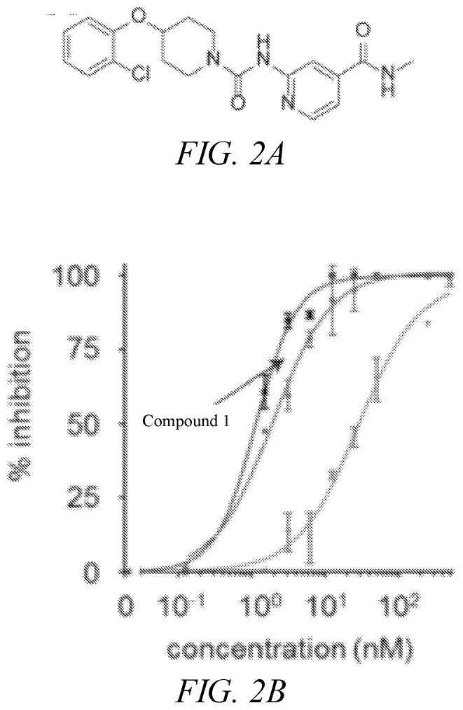

FIG. 2B shows competitive inhibition of SCD1 enzyme with Compound 1—an IC₅₀ equal to 1.9 nM is calculated using the curve indicated with the arrow.

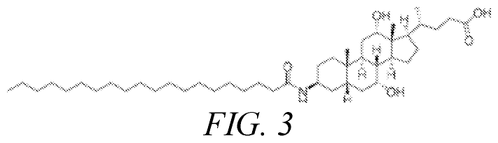

FIG. 3 shows Aramchol, a bile acid derivative of cholic acid reduces SCD1 mRNA expression levels and is currently in FDA clinical trials for several liver disease associated programs. However, high doses (600 mg) or twice daily (300 mg) treatment are needed. Aramchol serves as benchmark compound against Compound 1 in studies.

FIG. 4 shows how Compound 1 attenuates liver steatosis in mice fed on a Western Diet. C57BL6/J male mice were place on a Western Diet (WD) for 4 months and treated for 10 days IP with Compound 1 (2 mg/kg); FIG. 4A shows H&E staining (40×); FIG. 4B shows quantification of Compound 1 and significantly reduced liver steatosis (N=3. *P<0.05)

FIG. 5 shows results for treatment studies using Compound 1 showing improved liver function plasma markers. The study was performed with C57BL6/J male mice, placed on a Western Diet for 4 months, and treated for 10 days IP with Compound 1 (2 mg/kg−N=3; *P<0.05)

FIG. 6 shows toxicokinetic data of Compound 1 with maximum tolerated doses (FIGS. 6A-6D). Healthy beagles (Canis lupis familiaris) were dosed daily (oral gavage) for 14 days, and blood was collected on day 15. There were no significant changes in liver enzymes or cholesterol levels (or any other blood chemistry levels data no shown).

FIG. 6E shows no dramatic changes in WBC were noted. Preliminary data indicate efficacious doses for NASH are achieved at a 2 mg/kg and PK data is measured to delineate drug exposure levels at the efficacious doses tested. Compound 1 demonstrates a wide therapeutic window for the treatment of NASH.

### DETAILED DESCRIPTION

In the following description, certain specific details are set forth in order to provide a thorough understanding of various embodiments of the disclosure. However, one skilled in the art will understand that the disclosure may be practiced without these details.

Unless the context requires otherwise, throughout the present specification and claims, the word “comprise” and variations thereof, such as, “comprises” and “comprising” are to be construed in an open, inclusive sense, that is, as “including, but not limited to”.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, the terms “about” and “approximately” mean±20%, ±10%, ±5%, or ±1% of the indicated range, value, or structure, unless otherwise indicated. The terms “a” and “an” as used herein refer to “one or more” of the enumerated components. The use of the alternative (e.g., “or”) should be understood to mean either one, both, or any combination thereof of the alternatives.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the features, structures, or characteristics may be combined in any suitable manner in one or more embodiments.

Unless defined otherwise, all technical and scientific terms used herein have the same meaning as is commonly understood by one of skill in the art to which this disclosure belongs. As used in the specification and claims, the singular form “a”, “an” and “the” include plural references unless the context clearly dictates otherwise.

The term “effective amount” or “therapeutically effective amount” refers to that amount of a compound described herein that is sufficient to affect the intended application including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the compounds chosen, the dosing regimen to be followed, whether it is administered in combination with other compounds, timing of administration, the tissue to which it is administered, and the physical delivery system in which it is carried.

As used herein, “treatment” or “treating” refer to an approach for obtaining beneficial or desired results with respect to a disease, disorder or medical condition including but not limited to a therapeutic effect and/or a prophylactic effect. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. A prophylactic effect includes delaying or eliminating the appearance of a disease or condition, delaying or eliminating the onset of symptoms of a disease or condition, slowing, halting, or reversing the progression of a disease or condition, or any combination thereof. In certain embodiments, for prophylactic benefit, the compositions are administered to a subject at risk of developing a particular disease, or to a subject reporting one or more of the physiological symptoms of a disease, even though a diagnosis of this disease may not have been made.

The terms “co-administration,” “administered in combination with,” “further comprises administering a [therapeutic agent],” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

“Pharmaceutically acceptable salt” includes both acid and base addition salts.

“Pharmaceutically acceptable acid addition salt” refers to those salts which retain the biological effectiveness of the free bases, which are biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable acid addition salts are those that are pharmacologically effective and suitable for contact with the tissues of patients without undue toxicity, irritation, or allergic response.

Pharmaceutically acceptable acid addition salts which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness of the free acids, which are biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable base addition salts are those that are pharmacologically effective and suitable for contact with the tissues of patients without undue toxicity, irritation, or allergic response. Pharmaceutically acceptable base addition salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

In some embodiments, pharmaceutically acceptable salts include quaternary ammonium salts such as quaternary amine alkyl halide salts (e.g., methyl bromide).

The term “inhibitor” refers to a compound having the ability to inhibit a biological function of a target protein (e.g., an enzyme), whether by inhibiting the activity or expression of the protein, such as stearoyl-coenzyme A desaturase-1 (SCD1). Accordingly, the term “inhibitor” is defined in the context of the biological role of the target protein and include competitive inhibition (e.g., rather than downregulating mRNA expression).

The term “agonist” as used herein refers to a compound having the ability to initiate or enhance a biological function of a target protein, whether by inhibiting the activity or expression of the target protein. Accordingly, the term “agonist” is defined in the context of the biological role of the target protein (e.g., a PPAR-gamma agonist).

“Subject” refers to an animal, such as a mammal, for example a human. The methods described herein can be useful in both human therapeutics and veterinary applications. In some embodiments, the subject is a mammal, and in some embodiments, the subject is human.

“Mammal” includes humans and both domestic animals such as laboratory animals and household pets (e.g., cats, dogs, swine, cattle, sheep, goats, horses, rabbits), and non-domestic animals such as wildlife and the like.

“Prodrug” is meant to indicate a compound that may be converted under physiological conditions or by solvolysis to a biologically active compound described herein. Thus, the term “prodrug” refers to a precursor of a biologically active compound that is pharmaceutically acceptable. In some embodiments, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or thiol group is bonded to any group that, when the prodrug of the active compound is administered to a mammalian subject, cleaves to form a free hydroxy, free amino or free mercapto group, respectively. Examples of prodrugs include, but are not limited to, acetate, formate, and benzoate derivatives of a hydroxy functional group, or acetamide, formamide and benzamide derivatives of an amine functional group in the active compound and the like.

The term “in vivo” refers to an event that takes place in a subject's body.

Embodiments disclosed herein are also meant to encompass all pharmaceutically acceptable compounds, including salts, stereoisomers, tautomers, polymorphs, solvates, hydrates, isotopologues, and prodrugs thereof.

Certain embodiments are also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, embodiments include compounds produced by a process comprising administering a compound of this disclosure to a mammal for a period of time sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood or other biological samples.

A “pharmaceutical composition” refers to formulations of compounds of the disclosure and a medium generally accepted in the art for the delivery of compounds of the disclosure to mammals, e.g., humans. Such a medium includes all pharmaceutically acceptable carriers, diluents, or excipients therefor.

“Pharmaceutically acceptable carrier, diluent, or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier. In some embodiments, the pharmaceutical excipient is beta-cyclodextrin.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

The compounds of the disclosure (e.g., SCD1 inhibitors or PPAR-gamma agonists) or their pharmaceutically acceptable salts may contain one or more centers of geometric asymmetry and may thus give rise to stereoisomers such as enantiomers, diastereomers, and other stereoisomeric forms that are defined, in terms of absolute stereochemistry, as (R)- or (S)- or, as (D)- or (L)- for amino acids. Embodiments thus include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

Embodiments of the present disclosure include all manner of rotamers and conformationally restricted states of a compound of the disclosure. Atropisomers, which are stereoisomers arising because of hindered rotation about a single bond, where energy differences due to steric strain or other contributors create a barrier to rotation that is high enough to allow for isolation of individual conformers, are also included. As an example, certain compounds of the disclosure may exist as mixtures of atropisomers or purified or enriched for the presence of one atropisomer.

A “tautomer” refers to a proton shift from one atom of a molecule to another atom of the same molecule. Embodiments thus include tautomers of the disclosed compounds.

The chemical naming protocol and structure diagrams used herein are a modified form of the I.U.P.A.C. nomenclature system, using the ACD/Name Version 9.07 software program and/or ChemDraw Professional Version 17.0.0.206 software naming program (CambridgeSoft). For complex chemical names employed herein, a substituent group is typically named before the group to which it attaches. For example, cyclopropylethyl comprises an ethyl backbone with a cyclopropyl substituent. Except as described below, all bonds are identified in the chemical structure diagrams herein, except for all bonds on some carbon atoms, which are assumed to be bonded to sufficient hydrogen atoms to complete the valency.

### Compounds

It has been discovered that Compound 1 is a potent, reversible, and selective inhibitor of SCD1 with an IC₅₀ of 1.9 nM for blocking enzymatic activity, by directly measuring SCD1-catalyzed conversion of ¹³C₁₈-stearoyl CoA to ¹³C₁₈-oleoyl using LC-MS/MS in liver microsomes. Compound 1 does not significantly react off-target with any known kinases. Data show that Compound 1 reduces liver steatosis, and significantly improves liver function tests using a murine model fed with a Western Diet. Non-GLP pharmacokinetic (PK) and toxicokinetic (TK) studies in mice, rats and dogs show Compound 1 is well-tolerated and is orally bioavailable.

This disclosure provides identification of a pharmacologically active, potent, and selective SCD1 inhibitor (e.g., Compound 1) having good oral bioavailability that is amenable to oral formulations with small tablets/capsules, acceptable for daily dosing—a preferred clinical route of administration that does not require daily infusions.

In some embodiments, the SCD1 inhibitor is a compound of Structure (I):

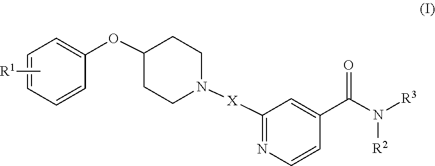

or a pharmaceutical salt, tautomer, stereoisomer, prodrug, or isotopologue thereof,
wherein:
   X is —(C═O)NR⁴—;
   R¹ is halo; and
   R², R³, and R⁴ are each independently hydrogen or an unsubstituted C₁₋₆ alkyl, to a subject in need thereof.

The disclosure provides compounds including pharmaceutically acceptable salts, tautomers, stereoisomers, isotopologues, and prodrugs thereof. In some embodiments, the compound is Compound 1. In some specific embodiments, the SCD1 inhibitor has the following structure:

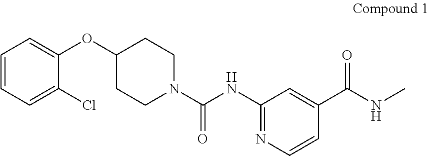 [SMILES: CNC(=O)c1ccnc(NC(=O)N2CCC(Oc3ccccc3Cl)CC2)c1]

or a pharmaceutical salt, tautomer, stereoisomer, prodrug, or isotopologue thereof.

In a more specific embodiment, the SCD1 inhibitor has the following structure:

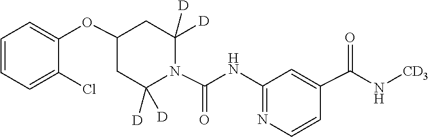 [SMILES: [2H]C([2H])([2H])NC(=O)c1ccnc(NC(=O)N2C([2H])([2H])CC(Oc3ccccc3Cl)CC2([2H])[2H])c1]

or a pharmaceutical salt, tautomer, stereoisomer, or prodrug thereof, wherein:
D is deuterium (i.e., ²H).

In some embodiments, SCD1 inhibitor has the following structure:

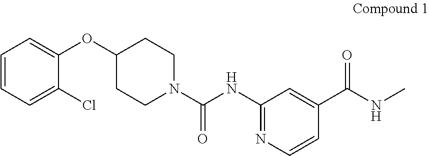 [SMILES: CNC(=O)c1ccnc(NC(=O)N2CCC(Oc3ccccc3Cl)CC2)c1]

or a pharmaceutical salt thereof.

In a more specific embodiment, the SCD1 inhibitor has the following structure:

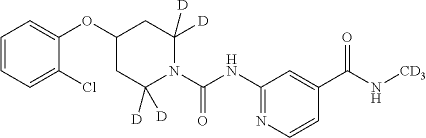 [SMILES: [2H]C([2H])([2H])NC(=O)c1ccnc(NC(=O)N2C([2H])([2H])CC(Oc3ccccc3Cl)CC2([2H])[2H])c1]

or a pharmaceutical salt thereof, wherein:
D is deuterium.

Some embodiments provide a compound having the following structure:

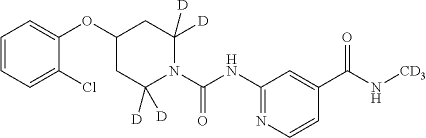 [SMILES: [2H]C([2H])([2H])NC(=O)c1ccnc(NC(=O)N2C([2H])([2H])CC(Oc3ccccc3Cl)CC2([2H])[2H])c1]

or a pharmaceutical salt thereof, wherein:
D is deuterium.

One embodiment provides a pharmaceutically acceptable salt of a compound having the following structure:

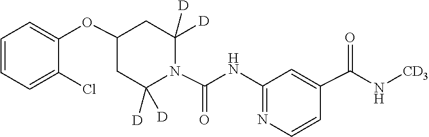

wherein:
D is deuterium.

Some embodiments provide a compound having the following structure:

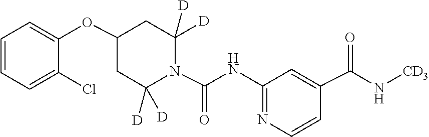

wherein:
D is deuterium.

Some embodiments provide a compound that is a PPAR-gamma agonist (e.g., pioglitazone). In some embodiments, the PPAR-gamma agonist has the following structure:

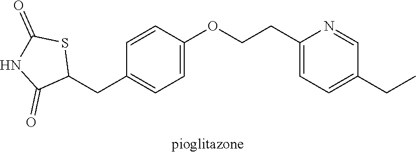 [SMILES: CCc1ccc(CCOc2ccc(CC3SC(=O)NC3=O)cc2)nc1]

or a pharmaceutical salt, tautomer, stereoisomer, or prodrug thereof.

In some embodiments, the PPAR-gamma agonist the PPAR-gamma agonist has the following structure:

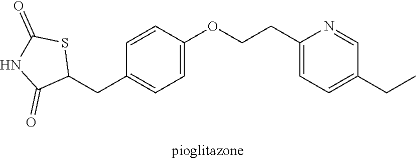 [SMILES: CCc1ccc(CCOc2ccc(CC3SC(=O)NC3=O)cc2)nc1]

or a pharmaceutical salt thereof. In some more specific embodiments, the pharmaceutically acceptable salt is an HCl, CF₃COOH, C₄H₄O₄, or (COOH)₂ salt. In more specific embodiments, the pharmaceutically acceptable salt is and HCl salt. In some embodiments, the pharmaceutically acceptable salt is a CF₃COOH. In some embodiments, the pharmaceutically acceptable salt is a C₄H₄O₄. In some embodiments, the pharmaceutically acceptable salt is a (COOH)₂ salt.

In an additional embodiment, various compounds of the disclosure (e.g., a compound of Structure (I) (e.g., Compound 1) or PPAR-gamma agonists) exist in free base or acid form can be converted to their pharmaceutically acceptable salts by treatment with the appropriate inorganic or organic base or acid by methods known to one skilled in the art. Salts of the compounds of the disclosure can be converted to their free base or acid form by standard techniques.

### Pharmaceutical Compositions

Other embodiments are directed to methods of administering or use of one or more pharmaceutical compositions. The pharmaceutical compositions comprise any one (or more) of a compound of Structure (I) (e.g., Compound 1) and/or a PPAR-gamma agonist and a pharmaceutically acceptable excipient or carrier. In some embodiments, the pharmaceutical composition is formulated for oral administration. In still more embodiments, the pharmaceutical compositions comprise a compound of Structure (I) (e.g., Compound 1) and an additional therapeutic agent. Examples of such therapeutic agents include agents that are PPAR-gamma agonists and/or Type-II diabetes drugs.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long-acting formulations are administered by implantation (for example subcutaneously or intramuscularly) or by intramuscular injection. Furthermore, in other embodiments, the compound is delivered in a targeted drug delivery system, for example, in a liposome coated with and organ-specific antibody. In such embodiments, the liposomes are targeted to and taken up selectively by the organ. In yet other embodiments, the compound as described herein is provided in the form of a rapid release formulation, in the form of an extended-release formulation, or in the form of an intermediate release formulation. In yet other embodiments, the compound described herein is administered topically.

In treatment methods according to embodiments of the disclosure, an effective amount of a compound of Structure (I) (e.g., Compound 1) and/or a PPAR-gamma agonist is administered to a subject suffering from or diagnosed as having such a disease, disorder, or medical condition. Effective amounts or doses may be ascertained by methods such as modeling, dose escalation studies, or clinical trials, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician.

A compound of Structure (I) (e.g., Compound 1) and/or PPAR-gamma agonists according to the disclosure are effective over a wide dosage range. For example, in the treatment of adult humans, dosages from 10 to 5000 mg, from 100 to 5000 mg, from 1000 mg to 4000 mg per day, and from 1000 to 3000 mg per day are examples of dosages that are used in some embodiments. The exact dosage will depend upon the route of administration, the form in which the compound is administered, the subject to be treated, the body weight of the subject to be treated, and the preference and experience of the attending physician.

In some embodiments, a compound of Structure (I) (e.g., Compound 1) and/or PPAR-gamma agonists of the disclosure are administered in a single dose. Administration can be oral or by injection, e.g., intravenous injection, in order to introduce the agent quickly. However, other routes are used as appropriate. A single dose of a compound of the disclosure may also be used for treatment of an acute condition.

In some embodiments, a compound of Structure (I) (e.g., Compound 1) and/or PPAR-gamma agonists of the disclosure are administered in multiple doses. In some embodiments, dosing is about once, twice, three times, four times, five times, six times, or more than six times per day. In other embodiments, dosing is about once a month, once every two weeks, once a week, or once every other day. In another embodiment, a compound of Structure (I) (e.g., Compound 1) and/or PPAR-gamma agonists are administered together about once per day up to and including 6 times per day. In another embodiment, the administration of a compound of Structure (I) (e.g., Compound 1) and/or PPAR-gamma agonists of the disclosure continues for less than about 7 days. In yet another embodiment the administration continues for more than about 6, 10, 14, 28 days, two months, six months, or one year. In some cases, continuous dosing is achieved and maintained as long as necessary.

Administration of a compound of Structure (I) (e.g., Compound 1) and/or PPAR-gamma agonists of the disclosure may continue as long as necessary. In some embodiments, a compound of Structure (I) (e.g., Compound 1) and/or PPAR-gamma agonists of the disclosure are administered for more than 1, 2, 3, 4, 5, 6, 7, 14, or 28 days. In some embodiments, a compound of Structure (I) (e.g., Compound 1) and/or PPAR-gamma agonists of the disclosure are administered for less than 28, 14, 7, 6, 5, 4, 3, 2, or 1 day. In some embodiments, a compound of Structure (I) (e.g., Compound 1) and/or PPAR-gamma agonists of the disclosure are administered chronically on an ongoing basis, e.g., for the treatment of chronic effects.

In some embodiments, the compound of Structure (I) (e.g., Compound 1) and/or PPAR-gamma agonists of the disclosure are administered in individual dosage forms. It is known in the art that due to inter-subject variability in compound pharmacokinetics, individualization of dosing regimen is necessary for optimal therapy.

In some embodiments, a compound of Structure (I) (e.g., Compound 1) and/or PPAR-gamma agonists described herein are formulated into pharmaceutical compositions. In specific embodiments, pharmaceutical compositions are formulated in a conventional manner using one or more physiologically acceptable carriers comprising excipients (e.g., beta-cyclodextrin) and auxiliaries which facilitate processing of the disclosed compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising a compound of Structure (I) (e.g., Compound 1) and/or PPAR-gamma agonists and pharmaceutically acceptable diluent(s), excipient(s), and carrier(s).

A pharmaceutical composition, as used herein, refers to a mixture of a compound of Structure (I) (e.g., Compound 1) and/or PPAR-gamma agonists with other chemical components, such as carriers, stabilizers, diluents, dispersing agents, suspending agents, thickening agents, and/or excipients. In certain embodiments, the pharmaceutical composition facilitates administration of a compound of Structure (I) (e.g., Compound 1) and/or PPAR-gamma agonists to an organism. In some embodiments, therapeutically effective amounts of a compound of Structure (I) (e.g., Compound 1) and/or PPAR-gamma agonists provided herein are administered in a pharmaceutical composition to a subject (e.g., a mammal) having a disease, disorder, or medical condition to be treated. In specific embodiments, the subject is a mammal. In a more specific embodiment, the subject is a human. In certain embodiments, therapeutically effective amounts vary depending on the severity of the disease, the age and relative health of the subject, the potency of a compound of Structure (I) (e.g., Compound 1) and/or PPAR-gamma agonists used and other factors. Compounds of Structure (I) (e.g., Compound 1) and/or PPAR-gamma agonists described herein are used singly or in combination with one or more therapeutic agents as components of mixtures.

In another embodiment, a compound of Structure (I) (e.g., Compound 1) and/or PPAR-gamma agonists are formulated for oral administration. Compounds described herein are formulated by combining the active compounds with, e.g., pharmaceutically acceptable carriers or excipients. In various embodiments, a compound of Structure (I) (e.g., Compound 1) and/or PPAR-gamma agonists are formulated in oral dosage forms that include, by way of example only, tablets, powders, pills, dragees, capsules, liquids, gels, syrups, elixirs, slurries, suspensions, and the like.

In certain embodiments, pharmaceutical preparations for oral use are obtained by mixing one or more solid excipient with a compound of Structure (I) (e.g., Compound 1) and/or PPAR-gamma agonists described herein, optionally grinding the resulting mixture, and processing the mixture of granules, after adding suitable auxiliaries, if desired, to obtain tablets or dragee cores. Suitable excipients are fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of a compound of Structure (I) (e.g., Compound 1) and/or PPAR-gamma agonists described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain a compound of Structure (I) (e.g., Compound 1) and/or PPAR-gamma agonists that are dissolved or suspended in a suitable liquid. Suitable liquids include, by way of example only, one or more fatty oil, liquid paraffin, or liquid polyethylene glycol. In addition, stabilizers are optionally added.

Pharmaceutical compositions include at least one pharmaceutically acceptable carrier, diluent, or excipient, and a compound of Structure (I) (e.g., Compound 1) and/or PPAR-gamma agonists described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of compounds of Structure (I) (e.g., Compound 1) and/or PPAR-gamma agonists described herein are included within the scope of the compounds presented herein. Additionally, compounds of Structure (I) (e.g., Compound 1) and/or PPAR-gamma agonists described herein encompass un-solvated as well as solvated forms with pharmaceutically acceptable solvents such as water, ethanol, and the like. The solvated forms of compounds of Structure (I) (e.g., Compound 1) and/or PPAR-gamma agonists presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising a compound of Structure (I) (e.g., Compound 1) and/or PPAR-gamma agonists described herein include formulating the compounds with one or more inert, pharmaceutically acceptable excipients or carriers to form a solid, semi-solid, or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions, and creams. The form of the pharmaceutical compositions described herein include liquid solutions or suspensions, solid forms suitable for solution or suspension in a liquid prior to use, or as emulsions. These compositions also optionally contain minor amounts of non-toxic, auxiliary substances, such as wetting or emulsifying agents, pH buffering agents, and so forth.

Compositions may include one or more antioxidants to enhance chemical stability where required. Suitable antioxidants include, by way of example only, ascorbic acid and sodium metabisulfite.

In certain embodiments, the formulations described herein comprise one or more antioxidants, metal chelating agents, thiol containing compounds, and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

In some embodiments, the concentration of a compound of Structure (I) (e.g., Compound 1) and/or PPAR-gamma agonists in the pharmaceutical compositions of the present disclosure is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of a compound of Structure (I) (e.g., Compound 1) and/or PPAR-gamma agonists provided in the pharmaceutical compositions of the present disclosure is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the amount a compound of Structure (I) (e.g., Compound 1) and/or PPAR-gamma agonists provided in the pharmaceutical compositions of the present disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of a compound of Structure (I) (e.g., Compound 1) and/or PPAR-gamma agonists provided in the pharmaceutical compositions of the present disclosure is in the range of 0.0001-10 g, 0.0005-9 g, 0.001-8 g, 0.005-7 g, 0.01-6 g, 0.05-5 g, 0.1-4 g, 0.5-4 g, or 1-3 g.

Packaging materials for use in packaging pharmaceutical compositions described herein include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more compounds described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound of Structure (I) (e.g., Compound 1) and/or PPAR-gamma agonists described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical compositions are presented in a pack or dispenser device which contains one or more unit dosage forms containing a compound provided herein. The pack for example contains metal or plastic foil, such as a blister pack. Or, the pack or dispenser device is accompanied by instructions for administration. Or, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound of Structure (I) (e.g., Compound 1) and/or PPAR-gamma agonists provided herein formulated in a compatible pharmaceutical carrier are prepared, placed in an appropriate container, and labeled for treatment of an indicated condition (e.g., NAFLD or NASH).

### Methods

Embodiments of the present disclosure generally relate to treatment of NAFLD and/or NASH via the inhibition of SCD1 in a host species. Related embodiments relate to method of reducing excess fat build up in the liver of a subject, the method comprising administering an effective amount of a compound of Structure (I) (e.g., Compound 1) to the subject in need thereof. Similarly, another embodiment provides a method for reducing the number of lipids in hepatic cells of a subject, the method comprising administering an effective amount of a compound of Structure (I) (e.g., Compound 1) to the subject in need thereof.

Some embodiments provide a method for reducing the number of lipids in a hepatic cell, the method comprising contacting the hepatic cell with an effective amount of a compound of Structure (I) (e.g., Compound 1).

Accordingly, one embodiment provides a method for treating non-alcoholic fatty liver disease, the method comprising administering an effective amount of a compound of Structure (I):

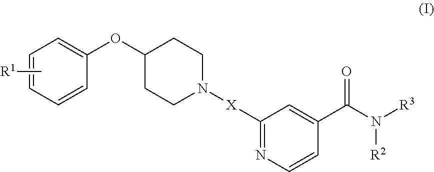

or a pharmaceutical salt, tautomer, stereoisomer, prodrug, or isotopologue thereof,
wherein:
   X is —(C═O)NR⁴—;
   R¹ is halo; and
   R², R³, and R⁴ are each independently hydrogen or an unsubstituted C₁₋₆ alkyl, to a subject in need thereof.

Another embodiment provides a method of treating non-alcoholic steatohepatitis, the method comprising administering an effective amount of a compound of Structure (I):

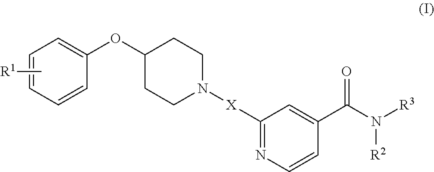

or a pharmaceutical salt, tautomer, stereoisomer, prodrug, or isotopologue thereof,
wherein:
   X is —(C═O)NR⁴—;
   R¹ is halo; and
   R², R³, and R⁴ are each independently hydrogen or an unsubstituted C₁₋₆ alkyl, to a subject in need thereof.

One embodiment provides a method of reducing excess fat build up in the liver of a subject, the method comprising administering an effective amount of a compound of Structure (I):

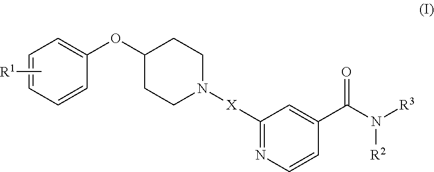

or a pharmaceutical salt, tautomer, stereoisomer, prodrug, or isotopologue thereof,
wherein:
   X is —(C═O)NR⁴—;
   R¹ is halo; and
   R², R³, and R⁴ are each independently hydrogen or an unsubstituted C₁₋₆ alkyl, to a subject in need thereof.

Yet another embodiment provides a method for reducing the number of lipids in hepatic cells of a subject, the method comprising administering an effective amount of a compound of Structure (I):

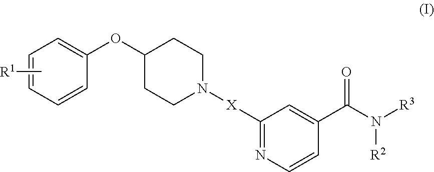

or a pharmaceutical salt, tautomer, stereoisomer, prodrug, or isotopologue thereof,
wherein:
   X is —(C═O)NR⁴—;
   R¹ is halo; and
   R², R³, and R⁴ are each independently hydrogen or an unsubstituted C₁₋₆ alkyl, to a subject in need thereof.

In some embodiments, the compound has the following Structure (Ia):

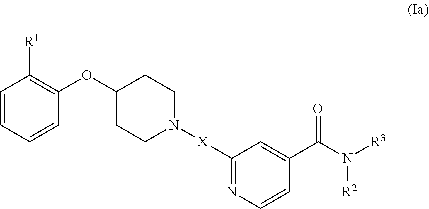

or a pharmaceutical salt, tautomer, stereoisomer, prodrug, or isotopologue thereof.

In some embodiments, R¹ is chloro. In certain embodiments, R⁴ is hydrogen. In some embodiments, R² is hydrogen. In certain embodiments, R³ is —CH₃. In some embodiments, the compound of Structure (I) is Compound 1 having the following structure:

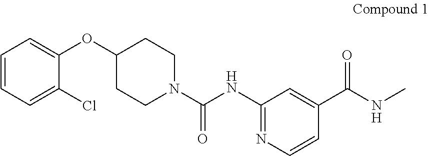 [SMILES: CNC(=O)c1ccnc(NC(=O)N2CCC(Oc3ccccc3Cl)CC2)c1]

or a pharmaceutical salt, tautomer, stereoisomer, prodrug, or isotopologue thereof, to a subject in need thereof.

In some embodiments, the method prevents or reduces inflammation of the liver. In certain embodiments, the method prevents or reduces liver fibrosis, cirrhosis, or scarring in the liver of the subject. In certain embodiments, the compound of Structure (I) is administered orally.

In some embodiments, the compound of Structure (I) is administered once per day. In certain embodiments, the compound of Structure (I) is administered once per day for at least 10 consecutive days. In some embodiments, the compound of Structure (I) is administered once per day for at least 14 consecutive days. In certain embodiments, the compound of Structure (I) is administered once per day for at least 21 consecutive days. In some embodiments, the compound of Structure (I) is administered once per day for at least 28 consecutive days.

In certain embodiments, mRNA levels of SCD1 are not downregulated. In some embodiments, mRNA levels of PPAR-gamma are upregulated. In certain embodiments, mRNA levels for fibrogenic genes are downregulated. In some embodiments, the level of reactive oxygen species is decreased. In some embodiments, the level of PPAR-gamma is increased.

In certain embodiments, the method further comprises administering a PPAR-gamma agonist. In some embodiments, the compound of Structure (I) is formulated with a pharmaceutical excipient. In certain embodiments, the compound of Structure (I) is formulated with beta-cyclodextrin.

In some embodiments, the method further comprises performing a clinical eye exam. In some embodiments, the method further comprises administering lipid eye drops. In some embodiments, the compound of Structure (I) and the PPAR-gamma agonist are formulated into a single dosage form comprising the compound of Structure (I) and the PPAR-gamma agonist. In some embodiments, the single dosage form further comprises a pharmaceutical excipient. In certain embodiments, the pharmaceutical excipient is beta-cyclodextrin.

In some embodiments, the PPAR-gamma agonist has the following structure:

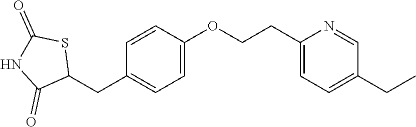 [SMILES: CCc1ccc(CCOc2ccc(CC3SC(=O)NC3=O)cc2)nc1]

or a pharmaceutical salt, tautomer, stereoisomer, or prodrug thereof.

In some embodiments, the PPAR-gamma agonist is a pharmaceutically acceptable salt of the following structure:

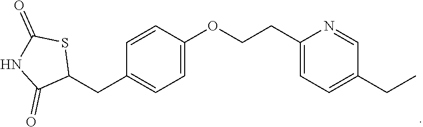 [SMILES: CCc1ccc(CCOc2ccc(CC3SC(=O)NC3=O)cc2)nc1]

In some embodiments, the pharmaceutically acceptable salt is an HCl salt.

In certain embodiments, the compound of Structure (I) is a deuterated form thereof. In certain embodiments, the compound of Structure (I) has the following structure:

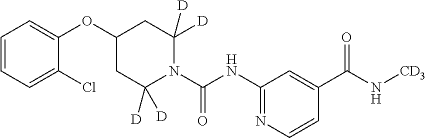 [SMILES: [2H]C([2H])([2H])NC(=O)c1ccnc(NC(=O)N2C([2H])([2H])CC(Oc3ccccc3Cl)CC2([2H])[2H])c1]

or a pharmaceutical salt, tautomer, stereoisomer, or prodrug thereof, wherein:
D is deuterium.

Some embodiments provide a method for reducing the number of lipids in a hepatic cell, the method comprising contacting the hepatic cell with an effective amount of a compound of Structure (I:

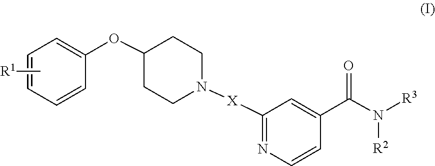

or a pharmaceutical salt, tautomer, stereoisomer, prodrug, or isotopologue thereof,
wherein:
   X is —(C═O)NR⁴—;
   R¹ is halo; and
   R², R³, and R⁴ are each independently hydrogen or an unsubstituted C₁₋₆ alkyl, to a subject in need thereof.

In some more specific embodiments, R¹ is chloro. In some embodiments, R⁴ is hydrogen. In certain embodiments, R² is hydrogen. In certain embodiments, R³ is —CH₃.

In some embodiments, the compound of Structure (I) is Compound 1 having the following structure:

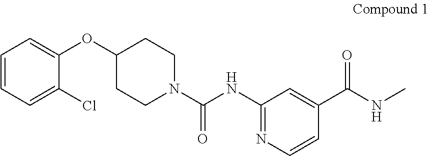 [SMILES: CNC(=O)c1ccnc(NC(=O)N2CCC(Oc3ccccc3Cl)CC2)c1]

or a pharmaceutical salt, tautomer, stereoisomer, prodrug, or isotopologue thereof, to a subject in need thereof.

In certain embodiments, the compound of Structure (I) is a competitive enzyme inhibitor. In some embodiments, mRNA levels of SCD1 are not downregulated in the hepatic cell. In certain embodiments, mRNA levels of PPAR-gamma are upregulated in the hepatic cell. In some embodiments, mRNA levels for fibrogenic genes are downregulated in the hepatic cell. In some embodiments, the level of reactive oxygen species is decreased in the hepatic cell. In some embodiments, the level of PPAR-gamma is increased in the hepatic cell. In some embodiments, the method further comprises contacting the hepatic cell with a PPAR-gamma agonist. In some embodiments, the PPAR-gamma agonist has the following structure:

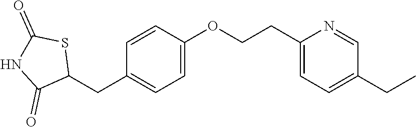 [SMILES: CCc1ccc(CCOc2ccc(CC3SC(=O)NC3=O)cc2)nc1]

or a pharmaceutical salt, tautomer, stereoisomer, or prodrug thereof.

In some embodiments, the compound of Structure (I) is a deuterated form thereof. In some embodiments, the compound of Structure (I) has the following structure:

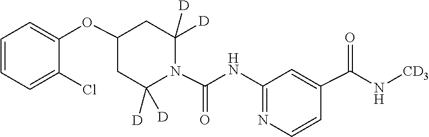 [SMILES: [2H]C([2H])([2H])NC(=O)c1ccnc(NC(=O)N2C([2H])([2H])CC(Oc3ccccc3Cl)CC2([2H])[2H])c1]

or a pharmaceutical salt, tautomer, stereoisomer, or prodrug thereof, wherein
D is deuterium.

The host, subject, or patient can belong to any mammalian species, for example a primate species, particularly humans; rodents, including mice, rats, and hamsters; rabbits; horses, cows, dogs, cats, etc. Animal models are of interest for experimental investigations, providing a model for treatment of human disease.

Furthermore, embodiments of the disclosure relate to the use of a compound of Structure (I) (e.g., Compound 1) and/or PPAR-gamma agonists and/or physiologically acceptable salts thereof to produce a medicament for the prophylactic or therapeutic treatment and/or monitoring of diseases that are caused, mediated, and/or modulated by NAFLD activity.

Embodiments of the methods of the disclosure can be performed either in vitro or in vivo. The susceptibility of a particular cell or group of cells to treatment with a compound of Structure (I) (e.g., Compound 1) and/or a PPAR-gamma agonist can be particularly determined by in vitro tests, whether during research or clinical application. Typically, a culture of a cell or cells is combined with a compound at various concentrations for a period which is sufficient to allow the active agents to inhibit SCD1 activity, usually between about one hour and one week. In vitro treatment can be carried out using cultivated cells from a biopsy sample or cell line.

Some embodiments described herein refer to “downregulation,” “upregulation,” “mRNA levels,” “level of [biological markers],” or similar. For these embodiments, the increase or decrease in expression is relative to control sample (e.g., other in vivo cells, a separate in vitro culture of cells, etc.) that has not received treatment. In some embodiments, the increase (e.g., upregulation) or decrease (e.g., downregulation) is a statistically significant difference. In some embodiments, the differences in levels or expression are measured for a group or sample of treated cells compared to a group or sample of untreated cells. In some embodiments, the differences in levels or expression are measured for a group or sample of treated subjects compared to a group or sample of untreated subjects.

### Compound Preparation

Compounds of Structure (I) (e.g., Compound 1) disclosed herein is known in the art and can be prepared according to methods known in the art. For example, synthesis of compounds of Structure (I) (e.g., Compound 1) can be found at least in PCT Publication No. WO 2016/022955, which is hereby incorporated by references for the details of that synthesis.

The PPAR-gamma agonists disclosed herein are those that are known in the art. In one embodiment the PPAR-gamma agonist has the following structure:

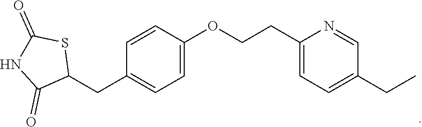 [SMILES: CCc1ccc(CCOc2ccc(CC3SC(=O)NC3=O)cc2)nc1]

In some embodiments, the foregoing PPAR-gamma agonist is an HCl, CF₃COOH, C₄H₄O₄, or (COOH)₂ salt. The foregoing PPAR-gamma agonist (and HCl, CF₃COOH, C₄H₄O₄, or (COOH)₂ salt thereof) can be prepared according to methods known in the art. For example, synthesis of the foregoing PPAR-gamma agonist and HCl, CF₃COOH, C₄H₄O₄, or (COOH)₂ salt thereof can be found at least in PCT Publication No. WO 2005/058827, which is hereby incorporated by references for the details of that synthesis.

### EXPERIMENTAL EXAMPLES

It was unexpectedly discovered that data was generated that shows pharmacological enzyme inhibition of SCD1 with Compound 1 leads to a reversal of liver steatosis in models of diet-induced liver steatosis of NAFLD/NASH. Without wishing to be bound by theory, it is thought that sustained inhibition of SCD1 with Compound 1 as a single agent attenuates hepatic lipogenesis, reduces triglyceride accumulation, and reduces liver injury. This can be shown by using diet-induced rodent models of NAFLD/NASH.

Test results to prove the above-noted aspects of the disclosure include a test to determine the therapeutic dose-range of Compound 1 in a diet-induced liver injury murine model.

Management or reversal of liver damage from NAFLD/NASH will impact an emerging leading cause of death worldwide. With a need for impactful pharmacotherapeutic management of NAFLD/NASH patients, the method of the present disclosure is (i.e., treatment with Compound 1) shows translational applicability to supporting patientcare in both the in-patient and out-patient clinical settings.

### Example 1

Treatment with Compound 1 as a Single Agent

This test determines NAFLD treatment for Compound 1. Inhibition of SCD1 enzyme activity Compound 1 attenuates the liver steatosis and metabolic dysfunction dose-dependently. Mice are fed either a Western high-fat Diet or a methionine-choline-deficient (MCD) diet to induce NAFLD/NASH for four months, to induce liver steatosis, and treated with different doses of Compound 1.

Reversal and/or attenuation of liver steatosis in Compound 1 treated mice is evaluated by histochemical and blood biomarkers. Data generated using the Western high fat diet is compared using a second model (MCD diet) that is more aggressive to determine whether inhibition reverses fibrotic disease. Single agent activity in both models is benchmarked to a more-broad spectrum SCD1 inhibitor Aramchol. In vitro studies with primary hepatocytes are used to validate mechanism of SCD1 inhibition and rigorously support the models.

To determine the therapeutic effectiveness of Compound 1 as SCD1 enzyme inhibitor to attenuate liver steatohepatitis in fed models of liver disease, classical diet induced murine model systems are used, with a reduction of clinically relevant lipid-associated liver damage with NAFLD/NASH. Numerous preclinical studies use these models to evaluate the in vivo efficacy of agents which attenuate liver injury.

Western high fat diet model: C57BL/6J mice are used, with both male and female mice included, aged 3 months. Mice are maintained on a standard chow diet and water ad libitum and housed in a room with a 12-hour light (6 am to 6 pm)/12-hour dark (6 pm to 6 am) cycle. Mice are randomly divided in 8 groups of with 6 males and 6 females based on our Power Analysis. Pair-fed control mice on standard chow normal diet are compared to mice fed on a Western high fat diet (HFD) (Harlan-Teklad 88137; 42% fat calories and 0.2% cholesterol). The mice are fed these diets for the study duration (16 weeks). In the last 2 weeks, Compound 1 is administered daily using a beta-cyclodextrin (Captisol) suspension vehicle with doses of 1 mg/kg, 2 mg/kg and 4 mg/kg orally. Aramchol is administered orally in comparison for benchmarking with dosing based on previously published literature, at 5 mg/kg/day (oral administration) for 2 weeks.

Methionine-choline-deficient (MCD) diet to induce NASH model: The classical mouse model of NASH is based on the MCD (Harlan-Teklad) which contains high sucrose (40%) and fat (10%) but is deficient in methionine and choline needed for hepatic mitochondrial beta-oxidation, as well as very low-density lipoprotein synthesis. This model leads to a more severe form of NASH with lipid peroxidation and fibrosis resembling those seen in human NASH. Mice are randomly divided in 8 groups of with 6 males and 6 females. Control mice are fed standard chow (Harlan-Tekland Control diet). The mice are fed these diets for the study duration (16 weeks). In the last 2 weeks, Compound 1 is administered using a beta-cyclodextrin (Captisol) suspension vehicle with doses of 1 mg/kg, 2 mg/kg and 4 mg/kg. Aramchol is administered orally as comparison for benchmarking based on previously published literature, with 5 mg/kg/day for 2 weeks.

Serum chemistry and histochemical analysis is done for both the Western Diet and MCD mouse models. Body weight and Echo-MRI is used to determine lean muscle, fat, and water content of the mice. At the end of the study, the animals are fasted overnight, and blood samples are collected for serum chemistry. A full panel of liver and metabolite biomarkers are assessed. This standard panel includes triglycerides and total cholesterol, triglycerides (TG), ALP, ALT, AST, Creatinine kinase, amylase, lipase, total bilirubin, LDH, as well as calcium, bicarbonate, potassium and sodium. Liver function tests from this panel include Aspartate aminotransferase level (AST), Alanine aminotransferase level (AST), GGT, Apolipoprotein, Glucose levels, and Alkaline Phosphatase (ALP), Proc-3, GAL-3, YKL-40, CK-18 and Caspase cleaved (MN-65, MN-30). Additionally, insulin, leptin and adiponectin levels are assessed as part of validation of models. Table 1 shows PK levels in dogs dosed at 20 mg/kg, rats dosed at 12.5 mg/kg, and mice dosed at 10 mg/kg by oral gavage (dog dose of 20 mg/kg adjusting BSA is 68.3 mg/kg in mice).

TABLE 1
Data related to dosing, route of administration,
Tₘₐₓ, Cₘₐₓ, AUC, and drug is plasma.

| | | | Sprague | |
|---|---|---|---|---|
| | | C57BL/6 Mice | Dawley Rat | Canine |
| Dose | | 10 mg/kg | 12.5 mg/kg | 20 mg/kg |
| Route of Administration | | Oral Gavage | Oral Gavage | Oral Gavage |
| Tₘₐₓ (hours) | | n/a | 0.5 | 1 |
| Cₘₐₓ (ng/mL) | | 4323 | 11,600 | 12,500 |
| AUCₗₐₛₜ (hour · ng/mL) | | 20,922 | 96,400 | 90,500 |
| Drug in | 4 hour | ~3,000 | 8,920 | 8,310 |
| Plasma | 8 hour | ~900 | 4,130 | 3,200 |
| (ng/mL) | 24 hour | ~25 | 437 | 212 |

### Example 2

Treatment with Compound 1 in Combination with a PPAR-Gamma Agonist

It is the aim of this study to determine the effect of combination therapy with Compound 1 and a PPAR-gamma agonist in a diet-induced liver injury murine model. Obesity and metabolic syndromes support the development of liver damage in NAFLD/NASH. The goal of this aim is to determine whether the combination treatment of Compound 1 with a PPAR-gamma agonist (i.e., pioglitazone) is superior to single agent treatment. The combination of Compound 1 with pioglitazone additively reduces liver injury and fibrosis, and reduces free fatty acids in the plasma, by attenuating a common co-morbid syndrome. A Western Diet-induced mouse model of NALFD is used in these studies.

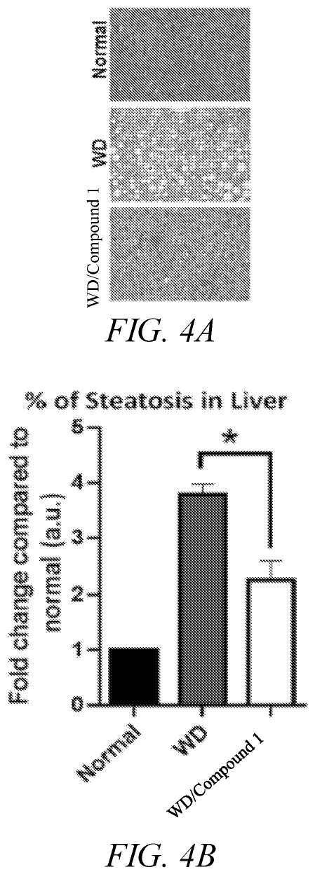

Without wishing to be bound by theory, it is believed that inhibition of SCD1 enzyme activity with Compound 1 attenuates hepatic lipogenesis, reduces triglyceride accumulation, and reduces liver injury in a diet-induced rodent model of NAFLD and fibrosis/NASH. Data is benchmarked against Aramchol (see, e.g., FIG. 3), a bile acid based SCD1 inhibitor in clinical trials (ARREST study; NCT02279524). Importantly the mechanism of action between Aramchol and Compound 1 is different; while Aramchol reduces expression of the mRNA for SCD1, Compound 1 inhibits enzymatic activity competitively and is more potent.

Both male and female mice (C75BL/6J) are used in studies because there is an indicated human difference in severity of liver injury between sexes for NAFLD/NASH. Compound 1 is expected to be equally effective in both sexes with attenuation of diet-induced liver damage.

A diet-induced liver injury murine model is used to determine the therapeutic dose-range for Compound 1. Studies are used to determine liver steatosis treatment using Compound 1. Without wishing to be bound by theory, it is thought that inhibition of SCD1 with Compound 1 attenuates liver steatosis and metabolic dysfunction dose-dependently, in mice fed with either a high fat diet or Western Diet.

This disclosure relates to a selective and nanomolar SCD1 inhibitor (Compound 1) that was developed using in silico modeling and high-throughput screening, with good oral bioavailability (F) and pharmacokinetic (PK) parameters for once-a-day dosing. Analytic methods have been developed and validated to detect Compound 1 in plasma and in tissues to ensure target engagement concentrations are reached. This will allow for the future development of human dosage forms which are smaller in size, as compared to the SCD1 modulator Aramchol (e.g., dosed at 600 mg dose).

Compared to the direct enzymatic SCD1 inhibition with Compound 1, Aramchol downregulates SCD1 as shown from mRNA levels.

### Example 3

Attenuation of Liver Stenatosis with Compound 1

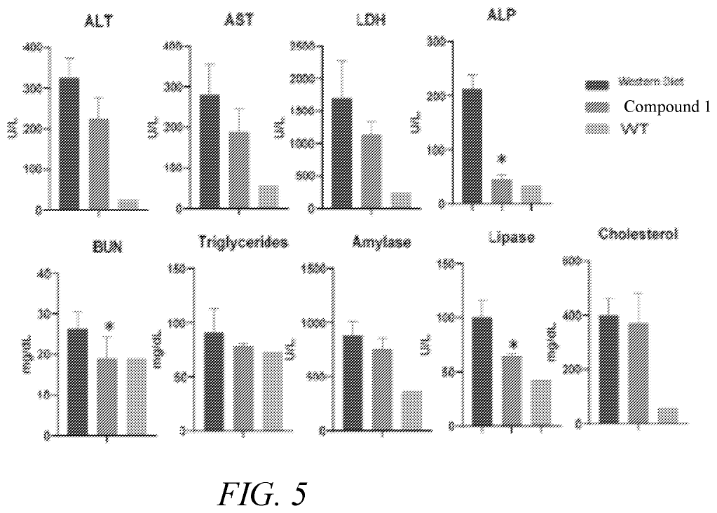

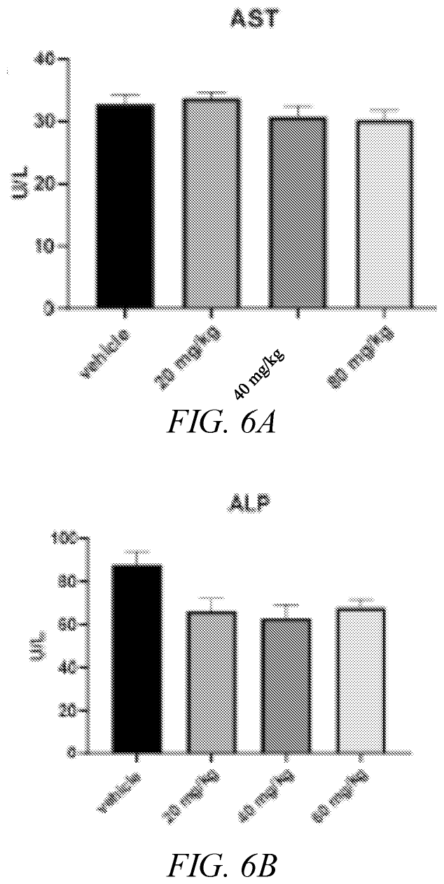

Studies with C57BL/6J male mice (N=3) placed on a four-month Western Diet and treated with Compound 1 at 2 mg/kg IP for 10 days show significant reduction in liver steatohepatitis (see, e.g., FIG. 4). Significant attenuation of liver damage was observed by studying serum chemistry, specifically ALP and BUN levels (see, e.g., FIG. 5).

### Example 4

### Oral Bioavailability and Dosing of Compound 1

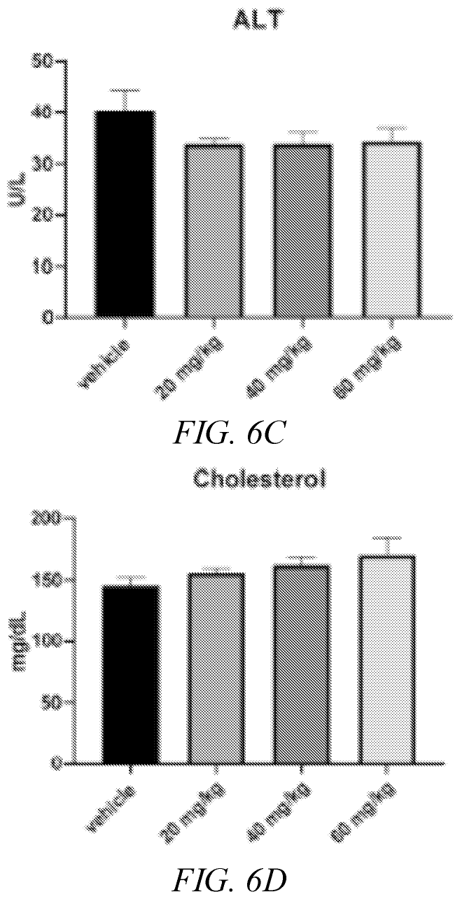

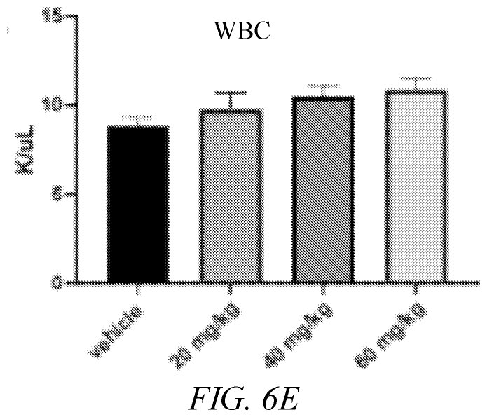

Compound 1 has a favorable oral bioavailability and safety profile (see, e.g., FIG. 6). Studies with both male and female rats indicated that Compound 1 is well tolerated when dosed orally. An oral dose range of 50-300 mg/kg was well tolerated in rats. Compound 1 does not induce liver enzymes in male and female rats, when dosed orally between 50-300 mg/kg, indicating a suitable safety profile.

A dosing regimen has been determined for Compound 1. Single dose and 7-day dosing non-GLP toxicity has been performed in rats and dogs and GLP toxicity in dogs 14 days repeat dosing. Toxicokinetic data show that the maximum tolerated dose (MTD) in dogs was found to be 20 mg/kg with Compound 1 treatment orally. The dose limiting toxicity for 14-day repeat dosing was due to ocular inflammation, when given at high doses. Dose conversion has been done via allometry based on body surface area.

Toxicology analysis (TK) of 14-day GLP dog study showed no substantial differences in chemistry, urinalysis, electrocardiograph, and hematological parameters compared to vehicle control ocular clinical observations were detected which are on target.

Ophthalmic observations of inflammation of the ocular surface with occasional corneal ulceration and low intraocular pressure in animals is observed when treated at the maximum tolerated doses, when compared to the control groups. These issues were attributed to atrophy of the Meibomian gland (the specialized sebaceous gland of the eyelid—see, e.g., FIG. 6). Most of the damage to eyes was reversible and not observed in the 14-day recover dogs (2M/2F). Mitigation the sequelae of events that occur due to dry eye is achieved by prophylactic administration of lipid eye drops and clinical eye exam every 14 days during a clinical trial.

### Example 5

### Liver Analysis

Livers are fixed in 4% paraformaldehyde, sectioned and evaluated using histochemical techniques, including H&E ultrastructure to measure fibrosis hepatocellular ballooning and necroinflammatory foci; lipid staining with Sirius Red, Sudan III, and CD64. Frozen sections are used to stain for lipid content with Oil Red O. Image analysis is done with ImageJ. Total RNA from mice livers is isolated by TRIzol, and a one-step Syber Green mRNA is extracted from the liver and evaluated by quantitative polymerase chain reaction (QPCR) for markers published from studies with Aramchol, including SCD1, fibrogenetic genes including COL1A1, ACTA2, bPDGFR2, MMP2, FXR, and PPAR-gamma. Western blot determines protein levels of Colala, αSMA, SCD1 and PPAR-gamma, and FXR and compared to Aramchol treatment.

### Example 6

### Plasma Concentration Analytics

Compound 1 quantification from mouse plasma is performed using LC-MS/MS on ExionLC system equipped with Kinetex XB-C₁₈, 2.1×50 mm (Phenomenex 00B-4496-AN) column coupled with AB Sciex 5500 QTrap instrument (equipped with TurboIonspray ionization source and Analyst software), with Acetonitrile/water/0.1% v/v formic acid as mobile phase. Blood samples collected are centrifuged at 1300 g, 2-8° C., ≥10 minutes and the collected plasma are stored either on ice (less than 1 hour) or at −80° C. until further processing. Predetermined quantities of plasma are vortexed with ice-cold acetonitrile to precipitate the protein content and centrifuged at 1300 g, 2-8° C., ≥10 minutes. Supernatant is taken in a labelled vial and evaporated at 40° C. under a gentle stream of nitrogen. The precipitates are reconstituted in LC-MS grade methanol and centrifuged at 1300 g, 2-8° C., ≥10 minutes. The supernatant is sampled for LC-MS/MS analysis. The concentration of Compound 1 is estimated by standard curve developed from plasma standards spiked with deuterium labeled Compound 1 as internal standard. Deuterium labeled Compound 1 is on-hand. In some embodiments, deuterium labeled Compound 1 has the following structure:

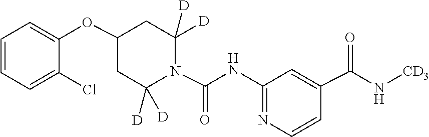 [SMILES: [2H]C([2H])([2H])NC(=O)c1ccnc(NC(=O)N2C([2H])([2H])CC(Oc3ccccc3Cl)CC2([2H])[2H])c1]

The extraction efficiency from mouse plasma and method is validated with low, medium, and high QC standards with appropriate blanks as needed.

### Example 7

### In Vitro Evaluation

Primary hepatocytes are used for in vitro evaluation as well as LX-2 human hepatic stellate cell line. The primary hepatocytes are isolated from the same mice cohort of C57BL/6J mice described above by collagenase perfusion. Cells are treated with various concentrations of Compound 1, while the positive control Aramchol is used at 10 μM for 48 hours. Cells are in either serum-free MEM or in an MCD rich medium. After 48 hours, the cells are incubated with BODIPY 493/503 (10 μg/mL) for 45 min prior to fixation with 4% paraformaldehyde, for quantification of lipid bodies with confocal microscopy.

For the cellular reactive oxygen species (ROS) determination, CellROX Deep Green is incubated at 1.5 μM for 30 min, and cell analyzed with flow cytometry. Similar protein and mRNA targets are compared to in vivo mouse data.

Without wishing to be bound by theory, it is expected that the mRNA for fibrogenic genes are downregulated, PPAR-gamma mRNA levels are upregulated, the mRNA levels of SCD1 will not be downregulated (an advantage over Aramchol), plasma markers for liver function is improved compared to the untreated mice, liver steatosis and fibrosis is attenuated to a greater extend as compared to Aramchol, with lower daily oral doses needed. It is expected that cell culture studies to recapitulate the mouse studies, with a decrease in the number of lipid bodies found in the hepatic cells, a decrease in ROS, and increase in beneficial PPAR-gamma mRNA levels are expected to be dose dependent for Compound 1. Lastly, it is not expected that there is a significant difference in plasma levels with the mice fed on the modified diets, since no significant change on serum albumin concentration was observed with Compound 1 treatment in the Western Diet mice.

### Example 7

### Alternative Dosing Regimen

The dosing period may be increased from 2 to 4 weeks with daily doses and evaluated based on the impact on liver steatosis. Compound 1 is evaluated in adipose tissue for beneficial effects of beiging on white adipose tissue (WAT), as alternative mechanism targeting the liver adipose tissue crosstalk. An alternative mouse model to evaluate the pharmacology of Compound 1 is the db/db diabetic dyslipidemia mouse model.

### Example 8

### Combination Treatment—Study 1

This study is used to determine the effect of combination therapy with Compound 1 in a diet induced liver injury murine model. Compound 1 is dosed in combination with pioglitazone, a type 2 diabetic drug and PPAR-gamma agonist. Obesity and metabolic syndromes support the development of liver steatosis, NAFLD/NASH. Meta-analysis found that pioglitazone is efficacious in human NAFLD, with off-label use. Pioglitazone is an FDA approved peroxisome proliferator-activated receptor (PPAR) gamma agonist, prescribed in the treatment of type II diabetes.

NAFLD/NASH treatment outcomes are improved when therapies are combined targeting lipid/triglyceride metabolism, insulin, and fibrotic metabolic contributions. These studies (see, e.g., FIG. 4 and FIG. 5) show that Compound 1 alone does not reduce plasma glucose or plasma triglycerides. C57BL/6J male and female mice aged 3 months are used for these studies. Mice are fed a Western High Fat Diet (WD) for a period of four months. In the last two weeks, mice are treated with oral gavaging. The treatment groups include:
1) Normal diet vehicle control
2) WD vehicle control
3) WD+pioglitazone (10 mg/kg)
4) WD+Compound 1 (2 mg/kg)
5) WD+Compound 1 (2 mg/kg)+pioglitazone (10 mg/kg).

Compound 1 and pioglitazone will made up in 20% w/v beta-cyclodextrin (Captisol). This suspension formulation for pioglitazone has been successfully used in long-term oral dosing in mice.

### Example 8

### Serum Chemistry and Histochemical Analysis—Combination Treatment

Serum chemistry and histochemical analysis is performed as described above with ultrastructure analysis of tissue ultrastructure to measure fibrosis including hepatocellular ballooning, necro-inflammatory foci, and lipids accumulation. Image analysis is done with ImageJ.

Without wishing to be bound by theory, it is expected that the attenuation of liver steatosis is significantly superior using a combination of pioglitazone with Compound 1 compared to single agent use. This improvement is in part due to the increased insulin sensitization, as measured by fasting glucose, insulin and decreased markers of liver injury determined by immunohistochemistry. We also expect that Compound 1 will not interfere with the pharmacology of pioglitazone.

Alternatively, compound treatment periods are be increased to 4 weeks for both Compound 1 and pioglitazone. A statin-based combination therapy with Compound 1 may be used to target lipid dysfunction and is impactful in liver disease.

Results are replicated to ensure reproducibility and guard against data bias. Several critical variables are selected to determine adequate sample size for studies. Meaningful differences (effect sizes) and standard deviation are determined, and a Power Analysis is conducted to determine differences in variables. For mouse studies, power analysis (G-power) indicates 6 mice per group per sex are necessary. A conservative sample size is selected to detect differences with an alpha level of 0.05 and 80% power. For histology, blinded evaluation by trained pathologists is done to ensure unbiased interpretation of data. Analysis is done using statistical software GraphPad Prism (most recent version), and ANOVA tests with appropriate post-hoc analysis, when comparing multiple independent groups, or a t-test when comparing two independent groups.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to U.S. Provisional Patent Application No. 63/356,419, filed Jun. 28, 2022, are incorporated herein by reference, in their entirety. Aspects of the embodiments can be modified, if necessary to employ concepts of the various patents, applications, and publications to provide yet further embodiments.

These and other changes can be made to the embodiments considering the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A method for treating non-alcoholic fatty liver disease, the method comprising administering an effective amount of a compound of Structure (I): 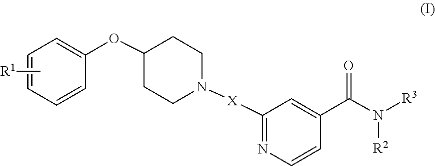
or a pharmaceutical salt, tautomer, stereoisomer, prodrug, or isotopologue thereof,
wherein:
X is —(C═O)NR⁴—;
R¹ is halo; and
R², R³, and R⁴ are each independently hydrogen or an unsubstituted C₁₋₆ alkyl, to a subject in need thereof.

2. A method of treating non-alcoholic steatohepatitis, the method comprising administering an effective amount of a compound of Structure (I): 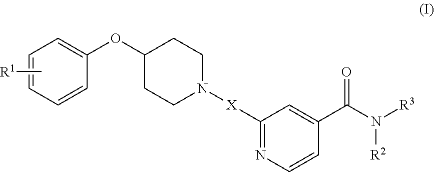
or a pharmaceutical salt, tautomer, stereoisomer, prodrug, or isotopologue thereof,
wherein:
X is —(C═O)NR⁴—;
R¹ is halo; and
R², R³, and R⁴ are each independently hydrogen or an unsubstituted C₁₋₆ alkyl, to a subject in need thereof.

3. A method of reducing excess fat build up in the liver of a subject, the method comprising administering an effective amount of a compound of Structure (I): 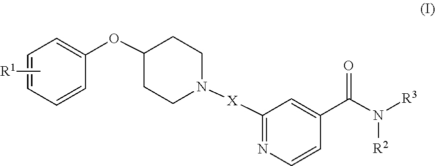
or a pharmaceutical salt, tautomer, stereoisomer, prodrug, or isotopologue thereof,
wherein:
X is —(C═O)NR⁴—;
R¹ is halo; and
R², R³, and R⁴ are each independently hydrogen or an unsubstituted CI-6 alkyl, to a subject in need thereof.

4. A method for reducing the number of lipids in hepatic cells of a subject, the method comprising administering an effective amount of a compound of Structure 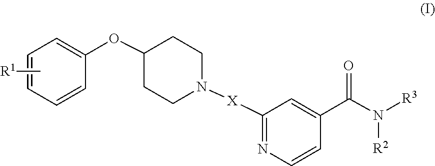
or a pharmaceutical salt, tautomer, stereoisomer, prodrug, or isotopologue thereof,
wherein:
X is —(C═O)NR⁴—;
R¹ is halo; and
R², R³, and R⁴ are each independently hydrogen or an unsubstituted CI-6 alkyl, to a subject in need thereof.

5. The method of any one of claims 1-4, wherein the compound has the following Structure (Ia): 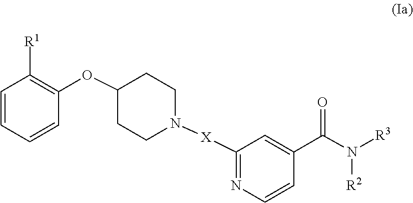
or a pharmaceutical salt, tautomer, stereoisomer, prodrug, or isotopologue thereof.

6. The method of any one of claims 1-5, wherein R¹ is chloro.

7. The method of any one of claims 1-6, wherein R⁴ is hydrogen.

8. The method of any one of claims 1-7, wherein R² is hydrogen.

9. The method of any one of claims 1-8, wherein R³ is —CH₃.

10. The method of any one of claims 1-9, wherein the compound of Structure (I) is Compound 1 having the following structure: 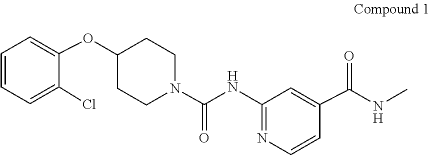 [SMILES: CNC(=O)c1ccnc(NC(=O)N2CCC(Oc3ccccc3Cl)CC2)c1]
or a pharmaceutical salt, tautomer, stereoisomer, prodrug, or isotopologue thereof, to a subject in need thereof.

11. The method of any one of claims 1-10, wherein the method prevents or reduces inflammation of the liver.

12. The method of any one of claims 1-11, wherein the method prevents or reduces liver fibrosis, cirrhosis, or scarring in the liver of the subject.

13. The method of any one of claims 1-12, wherein the compound of Structure (I) is administered orally.

14. The method of any one of claims 1-13, wherein the compound of Structure (I) is administered once per day.

15. The method of any one of claims 1-14, wherein the compound of Structure (I) is administered once per day for at least 10 consecutive days.

16. The method of any one of claims 1-15, wherein the compound of Structure (I) is administered once per day for at least 14 consecutive days.

17. The method of any one of claims 1-16, wherein the compound of Structure (I) is administered once per day for at least 21 consecutive days.

18. The method of any one of claims 1-17, wherein the compound of Structure (I) is administered once per day for at least 28 consecutive days.

19. The method of any one of claims 1-18, wherein mRNA levels of SCD1 are not downregulated.

20. The method of any one of claims 1-19, wherein mRNA levels of PPAR-gamma are upregulated.

21. The method of any one of claims 1-20, wherein mRNA levels for fibrogenic genes are downregulated.

22. The method of any one of claims 1-21, wherein the level of reactive oxygen species is decreased.

23. The method of any one of claims 1-22, wherein the level of PPAR-gamma is increased.

24. The method of any one of claims 1-23, wherein the method further comprises administering a PPAR-gamma agonist.

25. The method of any one of claims 1-24, wherein the compound of Structure (I) is formulated with a pharmaceutical excipient.

26. The method of any one of claims 1-25, wherein the compound of Structure (I) is formulated with beta-cyclodextrin.

27. The method of any one of claims 1-26, wherein the method further comprises performing a clinical eye exam.

28. The method of any one of claims 1-27, wherein the method further comprises administering lipid eye drops.

29. The method of any one of claims 1-28, wherein the compound of Structure (I) and the PPAR-gamma agonist are formulated into a single dosage form comprising the compound of Structure (I) and the PPAR-gamma agonist.

30. The method of claim 29, wherein the single dosage form further comprises a pharmaceutical excipient.

31. The method of claim 30, wherein the pharmaceutical excipient is beta-cyclodextrin.

32. The method of any one of claims 24-31, wherein the PPAR-gamma agonist has the following structure: 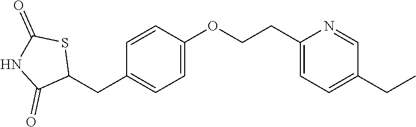 [SMILES: CCc1ccc(CCOc2ccc(CC3SC(=O)NC3=O)cc2)nc1]
or a pharmaceutical salt, tautomer, stereoisomer, or prodrug thereof.

33. The method of claim 24-32, wherein the PPAR-gamma agonist is a pharmaceutically acceptable salt of the following structure:

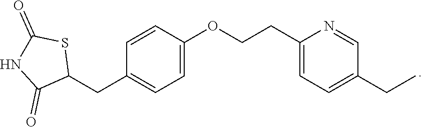 [SMILES: CCc1ccc(CCOc2ccc(CC3SC(=O)NC3=O)cc2)nc1]

34. The method of claim 33, wherein the pharmaceutically acceptable salt is an HCl salt.

35. The method of any one of claims 1-34, wherein the compound of Structure (I) is a deuterated form thereof.

36. The method of any one of claims 1-35, wherein the compound of Structure (I) has the following structure: 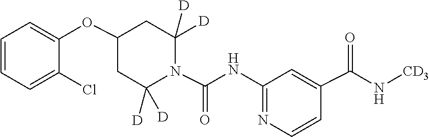 [SMILES: [2H]C([2H])([2H])NC(=O)c1ccnc(NC(=O)N2C([2H])([2H])CC(Oc3ccccc3Cl)CC2([2H])[2H])c1]
or a pharmaceutical salt, tautomer, stereoisomer, or prodrug thereof, wherein:
D is deuterium.

37. A method for reducing the number of lipids in a hepatic cell, the method comprising contacting the hepatic cell with an effective amount of a compound of Structure (I): 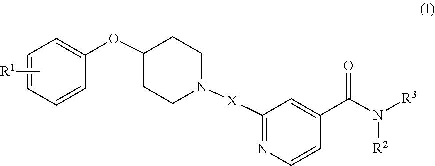
or a pharmaceutical salt, tautomer, stereoisomer, prodrug, or isotopologue thereof,
wherein:
X is —(C═O)NR⁴—;
R¹ is halo; and
R², R³, and R⁴ are each independently hydrogen or an unsubstituted C₁₋₆ alkyl, to a subject in need thereof.

38. The method of claim 37, wherein R¹ is chloro.

39. The method of any one of claims 37-38, wherein R⁴ is hydrogen.

40. The method of any one of claims 37-39, wherein R² is hydrogen.

41. The method of any one of claims 37-40, wherein R³ is —CH₃.

42. The method of any one of claims 37-41, wherein the compound of Structure (I) is Compound 1 having the following structure: 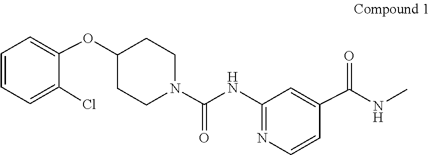 [SMILES: CNC(=O)c1ccnc(NC(=O)N2CCC(Oc3ccccc3Cl)CC2)c1]
or a pharmaceutical salt, tautomer, stereoisomer, prodrug, or isotopologue thereof, to a subject in need thereof.

43. The method of any one of claims 37-42, wherein the compound of Structure (I) is a competitive enzyme inhibitor.

44. The method of any one of claims 37-43, wherein mRNA levels of SCD1 are not downregulated in the hepatic cell.

45. The method of any one of claims 37-44, wherein mRNA levels of PPAR-gamma are upregulated in the hepatic cell.

46. The method of any one of claims 37-45, wherein mRNA levels for fibrogenic genes are downregulated in the hepatic cell.

47. The method of any one of claims 37-46, wherein the level of reactive oxygen species is decreased in the hepatic cell.

48. The method of any one of claims 37-47, wherein the level of PPAR-gamma is increased in the hepatic cell.

49. The method of any one of claims 37-48, wherein the method further comprises contacting the hepatic cell with a PPAR-gamma agonist.

50. The method of claim 49, wherein the PPAR-gamma agonist has the following structure: 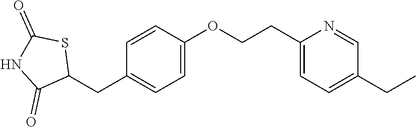 [SMILES: CCc1ccc(CCOc2ccc(CC3SC(=O)NC3=O)cc2)nc1]
or a pharmaceutical salt, tautomer, stereoisomer, or prodrug thereof.

51. The method of any one of claims 37-50, wherein the compound of Structure (I) is a deuterated form thereof.

52. The method of any one of claims 37-51, wherein the compound of Structure (I) has the following structure: 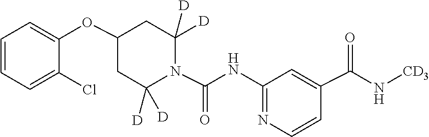 [SMILES: [2H]C([2H])([2H])NC(=O)c1ccnc(NC(=O)N2C([2H])([2H])CC(Oc3ccccc3Cl)CC2([2H])[2H])c1]
or a pharmaceutical salt, tautomer, stereoisomer, or prodrug thereof, wherein
D is deuterium.

53. A compound having the following structure: 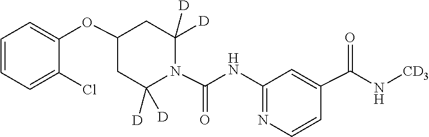 [SMILES: [2H]C([2H])([2H])NC(=O)c1ccnc(NC(=O)N2C([2H])([2H])CC(Oc3ccccc3Cl)CC2([2H])[2H])c1]
or a pharmaceutical salt, tautomer, stereoisomer, or prodrug thereof, wherein:
D is deuterium.

54. A compound having the following structure: 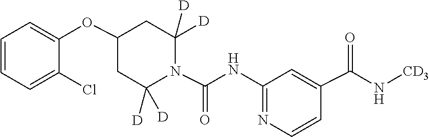 [SMILES: [2H]C([2H])([2H])NC(=O)c1ccnc(NC(=O)N2C([2H])([2H])CC(Oc3ccccc3Cl)CC2([2H])[2H])c1]
or a pharmaceutical salt thereof, wherein
D is deuterium.

55. A compound having the following structure: 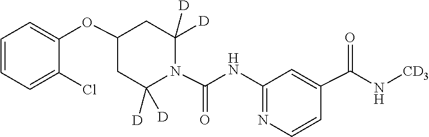
wherein:
D is deuterium.

56. A pharmaceutically acceptable salt of a compound having the following structure: 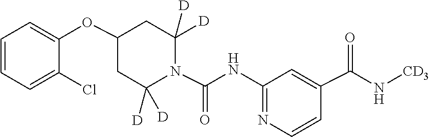
wherein:
D is deuterium.
